(19) United States
(12) Patent Application Publication
(10) Pub. No.: US 2011/0250297 A1
(43) Pub. Date: Oct. 13, 2011

(54) Treatment of Hyperproliferative Disorders Using Cardiac Glycosides

(76) Inventors: Bryan T. Oronsky, Los Altos Hills, CA (US); Peter Langecker, Monte Sereno, CA (US)

(21) Appl. No.: 13/120,678

(22) PCT Filed: Sep. 25, 2009
(86) PCT No.: PCT/US09/58491

§ 371 Date: Jun. 22, 2011

Related U.S. Application Data
(60) Provisional application No. 61/100,242, filed on Sep. 25, 2008.

(51) Int. Cl. A61K 33/06; A61K 33/00; A61P 1/00; A61P 9/00; A61P 19/02; A61K 31/7048; A61K 38/38

(52) U.S. Cl. 424/682; 514/26; 424/722; 514/15.2

(57) ABSTRACT

Provided are methods and compositions for treating and preventing hyperproliferative disorders such as psoriasis by administration of a cardiac glycoside alone or in combination locally or systemically with a calciotropic agents and/or a diffusion-limiting component, such a vasoconstrictor or collagen barrier.

### CROSS REFERENCE TO RELATED APPLICATIONS

This application claims the benefit of U.S. Provisional Appl. No. 61/100,242 filed Sep. 25, 2008, the disclosure of which is incorporated herein by reference.

### TECHNICAL FIELD

This application is related to methods for the treatment of proliferative disorders such as psoriasis with cardiac glycosides.

### BACKGROUND

The treatment of hyperproliferative disorders such as psoriasis is a constantly evolving field as new insights into the basic biology of these diseases has resulted in new therapeutic approaches. For example, the delineation of signal transduction pathways involved in the regulation of cell growth in both normal and hyperproliferative cells has provided new therapeutic targets for pharmacological intervention. The identification of cell surface proteins expressed on hyperproliferative cells, but not in their normally growing counterparts, has yielded further targets for therapeutic intervention using agents as such therapeutic antibodies. The fundamental discovery that many chemotherapeutic agents exert their cytotoxic effects by promoting programmed cell death or apoptosis has provided another mechanistic approach to the pharmacology of hyperproliferative disorders.

Given the ever expanding amount of knowledge on the basic biology of hyperproliferative disorders such as psoriasis, disorders of keratinization and keratosis, benign prostatic hypertrophy, diabetic retinopathy, endometriosis, macular degenerative disorders, hypertrophic scarring, cirrhosis, chronic inflammatory-related disorders, proliferative vitreoretinopathy, retinopathy of prematurity, granulomatosis, immune hyperproliferation associated with organ or tissue transplantation, an immunoproliferative disease or disorder, e.g., inflammatory bowel disease, rheumatoid arthritis, systemic lupus erythematosus (SLE), and vascular hyperproliferation secondary to retinal hypoxia or vasculitis, a number of different types of therapies are known. These include: surgery, chemotherapy, radiation therapy, immunotherapy, monoclonal antibody therapy, hormonal therapy, and inhibition of angiogenesis, among others. However, due to the heterogeneity of hyperproliferative cells, as well as, the development of resistance to anti-antiproliferative agents and therapies used during the course of treatment, new therapeutic approaches are always needed.

U.S. Patent Application No. 2006/0205679 relates to topical and oral formulations of cardiac glycosides for treating skin diseases. U.S. Pat. No. 6,071,885 is related to the treatment of PGF-mediated conditions by administration of a cardiac glycoside. U.S. Patent Application No. 2005/0026849 relates to water soluble formulations of digitalis glycosides for treating cell-proliferative and other diseases. PCT WO 00/47215 relates to methods antitumor therapy.

There remains a need for new approaches to the treatment of hyperproliferative diseases in general, and for improvements in the use of cardiac glycosides for the treatment of such diseases in particular. The present invention addresses these and other needs.

### BRIEF SUMMARY OF THE INVENTION

Provided are methods for treating hyperproliferative disorders, such as psoriasis, wound healing and cancer, using cardiac glycosides. Other examples of hyperproliferative disorders include: disorders of keratinization and keratosis, diabetic retinopathy, endometriosis, benign prostatic hypertrophy, macular degenerative disorders, hypertrophic scarring, cirrhosis, chronic inflammatory-related disorders, proliferative vitreoretinopathy, retinopathy of prematurity, granulomatosis, immune hyperproliferation associated with organ or tissue transplantation, an immunoproliferative disease or disorder, e.g., inflammatory bowel disease, rheumatoid arthritis, systemic lupus erythematosus (SLE), and vascular hyperproliferation secondary to retinal hypoxia or vasculitis. Other hyperproliferative disorders include cancer.

In some embodiments, contrary to standard practice, a cardiac glycoside is administered with a calciotropic agent, which elevates calcium levels, to potentiate the anti-proliferative effect of the cardiac glycoside. In yet other embodiments, the combination of a cardiac glycoside and a calciotropic agent, or a cardiac glycoside used alone, is administered at supra therapeutic doses in combination with a diffusion-limiting component, such as a vasoconstrictor or collagen barrier, to maintain localization of these pharmacological agents to regions of therapeutic interest. By localizing the pharmacological agents to a region of therapeutic interest, maximum and sustained therapeutic effect is achieved, while minimizing side effects to organs such as the heart. The compositions described herein may be applied to areas of patient's skin afflicted by a hyperproliferative disorder or may be administered via any suitable route including oral or systemic routes.

Accordingly, in one aspect, provided is a method of treating a hyperproliferative disorder in a subject by administering to the subject an anti-proliferation effective amount of a cardiac glycoside.

In another aspect, provided is a method of treating a hyperproliferative disorder in a subject by administering to the subject an anti-proliferation effective amount of a cardiac glycoside and at least one calciotropic agent, thereby treating the hyperproliferative disorder in the subject.

Also provided is a method of treating pain resulting from a hyperproliferative disorder in a subject by administering to the subject an anti-proliferation effective amount of a cardiac glycoside optionally in combination with a calciotropic agent.

Yet another aspect provides a method of treating pain resulting from a hyperproliferative disorder in a subject by administering to the subject an anti-proliferation effective amount of a cardiac glycoside and at least one calciotropic agent, thus treating the pain resulting from the hyperproliferative disorder in the subject.

A further aspect is a method for treating psoriasis in a subject by administering to the subject an anti-proliferation effective amount of a cardiac glycoside.

Another aspect is a method for treating cancer in a subject by administering an anti-proliferative effective amount of a cardiac glycoside optionally in combination with at least one calciotropic agent.

Another aspect is a method of treating a wound in a patient, the method comprising administering a cardiac glycoside optionally in combination with a calciotropic agent in effective amounts to treat a wound in the patient and promote healing.

Also provided is a pharmaceutical composition for the treatment of a hyperproliferative disorder, such as psoriasis or cancer, in a subject comprising an anti-proliferation effective amount of a cardiac glycoside and at least one calciotropic agent.

In a further aspect, provided is a pharmaceutical composition for the treatment of a hyperproliferative disorder in a subject comprising digoxin, epinephrine, and a collagen barrier.

Also provided is a kit for the treatment of a hyperproliferative disorder in which the kit comprises a cardiac glycoside, a calciotropic agent, and a diffusion limiting component.

In various embodiments of the above aspects, the hyperproliferative disorder is psoriasis, disorders of keratinization and keratosis, diabetic retinopathy, endometriosis, benign prostatic hypertrophy, macular degenerative disorders, keloids, warts, cirrhosis, chronic inflammatory-related disorders, proliferative vitreoretinopathy, retinopathy of prematurity, granulomatosis, immune hyperproliferation associated with organ or tissue transplantation, and an immunoproliferative disease or disorder. Examples of immunoproliferative disease or disorder include: inflammatory bowel disease, rheumatoid arthritis, systemic lupus erythematosus (SLE), vascular hyperproliferation secondary to retinal hypoxia, and vasculitis.

In some embodiments, the pain to be treated with the invention results from a hyperproliferative disorder including: psoriatic arthritis, rheumatoid arthritis, lupus, reactive arthritis, Sjogren's disease, inflammatory bowel disorder, dermatomyositis, ankylosing spondylitis, juvenile rheumatoid arthritis, gout, inflammatory osteoarthritis, pseudogout, and amyloidosis.

Cancers that can be treated include: acute lymphoblastic leukemia, acute myeloid leukemia, adrenocortical carcinoma, aids-related cancers, basal cell carcinoma, bladder cancer, bone cancer, brain stem glioma, breast cancer, bronchial tumors, burkitt lymphoma, central nervous system embryonal tumors, cerebellar astrocytoma, cervical cancer, leukemia, colon cancer, colorectal cancer, cutaneous t-cell lymphoma, endometrial cancer, ependymoblastoma, ependymoma, esophageal cancer, gallbladder cancer, gastric (stomach) cancer, gastrointestinal carcinoid tumor, gastrointestinal stromal tumor (gist), glioma, hairy cell leukemia, head and neck cancer, hepatocellular (liver) cancer, hodgkin lymphoma, hypopharyngeal cancer, hypothalamic and visual pathway glioma, laryngeal cancer, lip and oral cavity cancer, melanoma, mesothelioma, mouth cancer, multiple endocrine neoplasia syndrome, multiple myeloma, mycosis fungoides, myelodysplastic syndromes, myelodysplastic/myeloproliferative diseases, myelogenous leukemia, chronic, myeloid leukemia, adult acute, nasal cavity and paranasal sinus cancer, nasopharyngeal cancer, neuroblastoma, non-hodgkin lymphoma, non-small cell lung cancer, oral cancer, oropharyngeal cancer, ovarian cancer, ovarian cancer, pancreatic cancer, nasal cavity cancer, parathyroid cancer, breast cancer, prostate cancer, rectal cancer, renal cell (kidney) cancer, respiratory tract carcinoma, small cell lung cancer, small intestine cancer, testicular cancer, throat cancer, and thyroid cancer.

In some embodiments, the cardiac glycoside is administered locally or systemically. In another embodiment, the calciotropic agent and the cardiac glycoside is administered locally or systemically. In some embodiments, the calciotropic agent and the cardiac glycoside are administered locally at a location distal to a psoriatic lesion. In other embodiments the administration of the at least one calciotropic agent results in a transient or sustained rise in calcium levels.

The calciotropic agent is, e.g., Vitamin D3 (cholecalciferol), a Vitamin D3 analogue, PTH, lipid phosphatidylinositol, PTHrP, magnesium, thiazide diuretic, and lithium. A suitable vitamin D3 analogue is calcipotriene (Dovonex). Furthermore, examples of cardiac glycoside for use in the invention include: digoxin, digitoxin, medigoxin, lanatoside C, proscillaridin, K strophanthin, peruvoside, and ouabain.

In other embodiments, capsaicin or capsaicin congeners and derivatives are also administered. Examples of suitable capsaicin or capsaicin congeners and derivatives include: resiniferatoxin, Capsinolol, N-arachidonoyldopamine (NADA). Additional embodiments entail the further administration of at least one agent, such as a vasoconstrictor, a cell depolarizing agent, a pain-reducing agent, a chemotherapeutic agent, an anti-angiogenic agent, a radiosensitizer, a pain-reducing agent, a diffusion-limiting component, or calcium. An example of a suitable vasoconstrictor is epinephrine. Examples of pain-reducing agents include lidocaine, benzocaine, cetacaine, prilocalne, aniline cocaine, novocaine, or bupivicaine. In some embodiments, the administration of calcium results in a calcium concentration of about 1 to 50 mg/dl. Examples of anti-angiogenic agents include: angiostatins, VEGF inhibitors, such as Avastin, VEGF-trap, ONTAK, rhuMab-VEGF, endostatins, 2-methoxy-estradiol, thalidomide, and taxanes. Examples of radiosensitizers include: metronidazole (Flagyl), misonidazole, RO-07-0554, RO-11-3696, RO-03-8799 (Primonidazole), SR-2508 (Etanidazole), RSU-1069, bromodeoxyuridine, cisplatin, 5-fluorouracil, and taxanes. An example of a diffusion-limiting component is a collagen barrier.

In other embodiments, the cardiac glycoside is administered at a dose which is sub-therapeutic as compared to when the cardiac glycoside is administered alone. Examples of sub-therapeutic doses include those that result in a plasma concentration of less than 2.5 ng/ml for digoxin or less than 9-35 ng/ml for digitoxin.

In other embodiments, a cardiac glycoside is administered at a supra therapeutic dose. Examples of supra therapeutic doses include: at least 1.5 times greater or 3.0 times greater than a therapeutic dose for treatment of heart conditions. In further embodiments, a cardiac glycoside is administered at a standard therapeutic dose. In another embodiment, the cardiac glycoside is administered at a low dose.

The cardiac glycosides and other agents of the invention can be administered by any route, including but not limited to oral, inhalation, rectal, transdermal, ophthalmic, nasal, topical, vaginal, and parenteral administration. Examples of ophthalmic administration include intravitreal or intracameral. Examples of topical administration include buccal or sublingual. Examples of parenteral administration include: subcutaneous, intramuscular, intravenous, intradermal, intratracheal, or epidural. If parenteral administration is used, it can be performed via injection with a syringe or trocar.

### DETAILED DESCRIPTION OF THE INVENTION

### I. General

Cardiac glycosides are a class of natural products that have been traditionally used to increase cardiac contractile force in patients with congestive heart failure and cardiac arrhythmias. The most familiar members of this class of drugs include digoxin, digitoxin, and oubain, which are derived from the plant genera Digitalis and Strophanthus gratus, respectively. Their mechanisms of action in the heart involve inhibition of the plasma membrane Na⁺/K⁺-ATPase, leading to increased intracellular Na⁺ and Ca⁺² and decreased intracellular K⁺. The increased intracellular Ca⁺² promotes muscle contraction and cardiac contractile force.

As a consequence of this mechanism of action, cardiac glycoside toxicity may occur at elevated doses of these drugs because prolonged inhibition of the Na⁺/K⁺-ATPase leads to the excessive loading of cardiac muscle cells with calcium. The overloading of cardiac muscle cells can then lead to life threatening ventricular tachycardia followed by ventricular fibrillation. Because calcium is known to potentiate the toxicity of cardiac glycosides, it is generally accepted that hypercalcemia predisposes a patient to cardiac glycoside toxicity. For this reason, hypercalcemic conditions are avoided in patients receiving cardiac glycoside treatment.

Cardiac glycosides have been described for the treatment of cancer. The anti-tumor effects of cardiac glycosides has been attributed to the ability of these compounds to induce calcium influx and resultant cell death or apoptosis. However, a shortcoming associated with the use of cardiac glycosides for the treatment of cancer is the possibility of calcium overloading of cardiac muscle cells, and hence, heart function irregularities, especially when elevated doses of these agents are used.

The use of calciotropic agents combined with the administration of sub-therapeutic doses of cardiac glycoside can provide an effective anti psoriasis treatment while avoiding cardiac glycoside toxicity because lower than cardiotoxic doses of these drugs may be used. This result is unexpected, because the conventional wisdom in the art of cardiac glycoside pharmacology was to avoid the use of these drugs under conditions that could result in hypercalcemia.

Also provided are methods for the administration of supra therapeutic doses of cardiac glycosides for the treatment of hyperproliferative disorders such as psoriasis by teaching methods to minimize diffusion of these agents from therapeutically relevant locations on the body, thus avoiding potentially life threatening side effects in non-therapeutically relevant locations such as the heart.

### II. Definitions

The term “treating” a hyperproliferative disorder refers to any method which has the effect of slowing, retarding, or reversing the progression of a hyperproliferative disease or one or more symptoms or conditions associated with these diseases in a subject. The term also refers to methods which prevent or delay the onset of hyperproliferative disorders or one or more symptoms or conditions associated with this disease. Accordingly, the term “treating” may be used interchangeably with “amelioriating,”, “reducing”, or “inhibiting”. Thus, “treating” may include killing, inhibiting or slowing the growth or increase in size of a body or population of hyperproliferative cells, reducing hyperproliferative cell numbers, or preventing spread to other anatomic sites, as well as reducing the size of a hyperproliferative growth or numbers of hyperproliferative cells.

The term “an anti-proliferation effective amount” refers to the amount of an anti-proliferation agent which is effective in treating a hyperproliferative disorder. An “anti-proliferation effective amount” will vary depending on therapeutic regime. Thus, for example, the anti-proliferation effective amount of an anti-psoriasis agent may be higher when used alone as compared to the anti-proliferation effective amount when that agent is used with a potentiating agent, e.g., when a cardiac glycoside is used with a calciotropic agent as described herein.

A “subtherapeutic dose” refers to a dose of a pharmacologically active agent that is functionally insufficient to elicit an intended pharmacological effect by itself (e.g., a psoriasis treatment), or that is quantitatively less than the established therapeutic dose for that particular pharmacological agent (e.g., as published in a reference such as the Physicians' Desk Reference, 62nd Ed., 2008, Thomson Healthcare or Brunton, et al., Goodman & Gilman's The Pharmacological Basis of Therapeutics, 11th edition, 2006, McGraw-Hill Professional). A “subtherapeutic dose” can be defined in relative terms (i.e., as a percentage amount (less than 100%) of the amount of pharmacologically active agent conventionally administered). For example, a subtherapeutic dose amount can be about 1% to about 25% of the amount of pharmacologically active agent conventionally administered. In some embodiments, a subtherapeutic dose can be about 1%, 2%, 3%, 5%, 10%, 12%, 15%, 20%, or 25% of the amount of pharmacologically active agent conventionally administered.

Alternatively, a “subtherapeutic dose” is one that results in blood levels of a pharmacological agent which is lower, either systemically or locally, than that obtained when an established therapeutic dose for that particular pharmacological agent is administered. Accordingly, a “subtherapeutic dose” can result from the administration of a pharmacological agent at a lower than established dosage, or via a route or dosing schedule different from an established therapeutic dosage or administration protocol, as discussed below.

A “supra therapeutic dose” refers to a dose of a pharmacologically active agent that is quantitatively greater than the established therapeutic dose for that particular pharmacological agent (e.g., as published in a reference such as those cited above) or to a dose which typically leads to undesirable side effects when used alone. A “supra therapeutic dose” can be defined in relative terms (i.e., as a percentage amount (greater than 100%) of the amount of pharmacologically active agent conventionally administered). For example, a supra therapeutic dose amount can be about 101% to about 500% of the amount of pharmacologically active agent conventionally administered. In some embodiments, a supra therapeutic dose can be about 125%, 150%, 175%, 200%, 250%, 300%, 400%, or 500% of the amount of pharmacologically active agent conventionally administered.

Alternatively, a “supra therapeutic dose” is one that results in blood levels of a pharmacological agent which is higher, either systemically or locally, than that obtained when an established therapeutic dose for that particular pharmacological agent is administered. Accordingly, a “supra therapeutic dose” can result from the administration of a pharmacological agent in a higher than established dosage, or via a route or dosing schedule different from an established therapeutic dosage or administration protocol, as discussed below.

A “standard” therapeutic dose refers to a dose of a pharmacologically active agent that is quantitatively the same as the established therapeutic dose for that particular pharmacological agent (e.g., as published in a reference such as those cited above) or to a dose which typically does not lead to undesirable side effects when used alone. Thus, in the case of cardiac glycosides, the standard therapeutic dose would be that normally used to treat heart conditions, such as congestive heart failure and cardiac arrhythmias. As discussed in greater detail below, cardiac glycosides are typically administered for the treatment of heart conditions in a first loading dose (digitization), followed by a maintenance dose.

“Psoriasis” refers to a hyperproliferative disorder which affects the skin and joints. Psoriasis commonly causes red scaly patches to appear on the skin, termed psoriatic plaques, which are areas of inflammation and excessive skin production. In this disease, skin rapidly accumulates at these sites and takes a silvery-white appearance. Plaques frequently occur on the skin of the elbows and knees, but can affect any area including the scalp and genitals. The forms of psoriasis include: plaque psoriasis (psoriasis vulgaris) (L40.0); flexural psoriasis (inverse psoriasis) (L40.83-4); guttate psoriasis (L40.4); pustular psoriasis (L40.1-3, L40.82); nail psoriasis (L40.86); psoriatic arthritis (L40.5); and erythrodermic psoriasis.

The term “cardiac glycoside” refers to a class of pharmacological agents including those that have been used to treat congestive heart failure and heart arrhythmias by inhibiting the Na+/K+ pump in cells. Inhibition of the Na+/K⁺ pump by cardiac glycosides leads to increased Na⁺ levels, which in turn slows down the extrusion of Ca⁺² via the Na⁺/Ca⁺² exchange pump. Many cardiac glycosides are natural products which share a common molecular motif comprising a steroid nucleus containing an unsaturated lactone ring at the C₁₇ position and one or more glycosidic residues at C₃. Examples of cardiac glycosides include, but are not limited to, ouabain, oleandrin, g/k/e-strophanthin, digoxin, digitoxin, proscillaridine A, which are plant derived, and bufalin, marinobufagenin and bufadienolides, which are derived from frog poisons. Cardiac glycosides comprise two structural features, a sugar (glycoside) and a non-sugar (aglycone) steroid moiety.

The term “calciotropic” as used herein refers to an agent that is involved in the regulation of calcium levels. Thus, a “calciotropic” agent can either increase, decrease, or modulate calcium levels, either systemically, locally, extracellularly, or intracellularly.

A “diffusion-limiting component” refers to an agent which serves to minimize the distribution or diffusion of the pharmacological agents of the invention from a localized region of the body, generally from a region of the body where a therapeutic effect is desired. Examples of diffusion-limiting components include vasoconstrictors, which limit distribution via the vascular system, or physical barriers, such as collagen barriers.

### III. Methods of Treating or Preventing Hyperproliferative Disorders using Cardiac Glycosides

1) Conditions Subject to Treatment

The present methods and compositions find use in the treatment and prevention of hyperproliferative disorders, particularly psoriasis.

In particular, the present methods and compositions find use in the slowing, retarding, or reversing the progression of psoriasis or one or more symptoms or conditions associated with the disease in a subject. Accordingly, the present invention also provides for the amelioration, inhibition, reduction, or prevention of symptoms indicative of psoriasis, as described herein.

Other examples of hyperproliferative disorders that may be treated using the methods of the invention include, but are not limited to, hyperproliferative arterial stenosis, inflammatory arthritis, hyperkeratoses and papulosquamous eruptions including arthritis. Also included are viral induced hyperproliferative diseases such as warts and EBV induced disease (i.e., infectious mononucleosis), scar formation, and the like.

The compounds and compositions disclosed herein also can be used for the treatment of wounds, to promote healing, as well as to alleviate dry skin conditions.

The compounds and compositions disclosed herein also can be used for the treatment of pain resulting from a hyperproliferative disorder in a subject. Thus, a method is provided that includes administering to a subject an anti-proliferation effective amount of a cardiac glycoside and optionally at least one calciotropic agent to alleviate pain in the subject. The hyperproliferative disorder is e.g., psoriatic arthritis, rheumatoid arthritis, lupus, reactive arthritis, Sjogren's disease, inflammatory bowel disorder, dermatomyositis, ankylosing spondylitis, juvenile rheumatoid arthritis, gout, inflammatory osteoarthritis, pseudogout, and amyloidosis.

Cancers that can be treated include: acute lymphoblastic leukemia, acute myeloid leukemia, adrenocortical carcinoma, aids-related cancers, basal cell carcinoma, bladder cancer, bone cancer, brain stem glioma, breast cancer, bronchial tumors, burkitt lymphoma, central nervous system embryonal tumors, cerebellar astrocytoma, cervical cancer, leukemia, colon cancer, colorectal cancer, cutaneous t-cell lymphoma, endometrial cancer, ependymoblastoma, ependymoma, esophageal cancer, gallbladder cancer, gastric (stomach) cancer, gastrointestinal carcinoid tumor, gastrointestinal stromal tumor (gist), glioma, hairy cell leukemia, head and neck cancer, hepatocellular (liver) cancer, hodgkin lymphoma, hypopharyngeal cancer, hypothalamic and visual pathway glioma, laryngeal cancer, lip and oral cavity cancer, melanoma, mesothelioma, mouth cancer, multiple endocrine neoplasia syndrome, multiple myeloma, mycosis fungoides, myelodysplastic syndromes, myelodysplastic/myeloproliferative diseases, myelogenous leukemia, chronic, myeloid leukemia, adult acute, nasal cavity and paranasal sinus cancer, nasopharyngeal cancer, neuroblastoma, non-hodgkin lymphoma, non-small cell lung cancer, oral cancer, oropharyngeal cancer, ovarian cancer, ovarian cancer, pancreatic cancer, nasal cavity cancer, parathyroid cancer, breast cancer, prostate cancer, rectal cancer, renal cell (kidney) cancer, respiratory tract carcinoma, small cell lung cancer, small intestine cancer, testicular cancer, throat cancer, and thyroid cancer.

2) Pharmacological Agents

The pharmacological agents used in the present methods and compositions include the one or more active agents, described in detail below, in any pharmaceutically acceptable form, including any pharmaceutically acceptable salts, prodrugs, racemic mixtures, conformational and/or optical isomers, crystalline polymorphs and isotopic variants of the one or more pharmacological agents.

In some embodiments, provided are methods to treat or prevent hyperproliferative disorders in a subject by administering to an individual in need thereof an anti-antiproliferation effective amount of a cardiac glycoside alone or in combination with one or more calciotropic agents. In other embodiments, the combination of the cardiac glycoside and calciotropic agents are administered with a further agent that minimizes diffusion of these agents to a location of the body in need of therapy. Examples of such diffusion-limiting components include vasoconstrictors, e.g., epinephrine, and collagen barriers. In other embodiments, supra therapeutic doses of a cardiac glycoside are used with a diffusion-limiting component in the absence of a calciotropic agent. Through the use of a diffusion-limiting component, the adverse effects of cardiac glycosides on non-target organs such as the heart can be minimized.

A. Cardiac Glycosides

Digoxin, also known as digitalis, is a purified cardiac glycoside extracted from the foxglove plant, Digitalis lanata. The systematic name for this compound is 4-[(3S,5R,8R,9S,10S,12R,13S,14S)-3-[(2S,4S,5R,6R)-5-[(2S,4S,5R,6R)-5-[(2S,4S,5R,6R)-4,5-dihydroxy-6-methyl-oxan-2-yl]oxy-4-hydroxy-6-methyl-oxan-2-yl]oxy-4-hydroxy-6-methyl-oxan-2-yl]oxy-12,14-dihydroxy-10,13-dimethyl-1,2,3,4,5,6,7,8,9,11,12,15,16,17-tetradecahydrocyclopenta[a]phenanthren-17-yl]-5H-furan-2-one. Digoxin preparations are commonly marketed under the trade names: Lanoxin, Digitek, and Lanoxicaps. Commercially available dosage forms include a 0.05 mg/mL oral solution and 0.25 mg/mL or 0.5 mg/mL injectable solution. Digoxin is usually administered orally, but it can also be administered by IV injection in some situations. When IV injection is used, the administration should be should be slow, and heart rhythm should be monitored.

The occurrence of adverse drug reactions is common, owing to its narrow therapeutic index (the margin between effectiveness and toxicity). Adverse effects are concentration-dependent. When used alone, adverse reactions are rare when plasma digoxin concentration is <0.8 μg/L. Common adverse effects (=1% of patients) include: loss of appetite, nausea, vomiting, diarrhea, blurred vision, visual disturbances (yellow-green halos), confusion, drowsiness, dizziness, nightmares, agitation, and/or depression. Less frequent adverse effects (0.1%-1%) include: acute psychosis, delirium, amnesia, shortened QRS complex, atrial or ventricular extrasystoles, paroxysmal atrial tachycardia with AV block, ventricular tachycardia or fibrillation. The pharmacological actions of digoxin usually results in electrocardiogram (ECG) changes, including ST depression or T wave inversion, which do not indicate toxicity. PR interval prolongation, however, may be a sign of digoxin toxicity. Additionally, increased intracellular Ca2+ may cause a type of arrhythmia called bigeminy (coupled beats), eventually ventricular tachycardia or fibrillation. The combination of increased (atrial) arrhythmogenesis and inhibited atrio-ventricular conduction (for example paroxysmal atrial tachycardia with A-V block—so-called “PAT with block”) is said to be pathognomonic (i.e. diagnostic) of digoxin toxicity.

To counteract adverse reactions, for example the occurrence of arrhythmias or malignant hyperkalaemia, the specific antidote of antidigoxin (antibody fragments against digoxin, under the trade names of Digibind and Digifab) may be used. Toxicity can also be treated with higher than normal doses of potassium.

Digitoxin is a cardiac glycoside which is the corresponding aglycone of digoxin. Thus, it has the systematic name: (3β,5β)-3-[(O-2,6-dideoxy-β-D-ribo-hexapyranosyl-(1->4)-2,6-dideoxy-β-D-ribo-hexopyranosyl)oxy]-14-hydroxycard-20(22)-enolide. Digitoxin has longer-lasting effects than digoxin because unlike digoxin (which is eliminated from the body via the kidneys), digitoxin is eliminated via the liver. Thus, it can be used in patients with poor or erratic kidney function. Digitoxin exhibits similar toxic side effects as the more-commonly used digoxin, namely: anorexia, nausea, vomiting, diarrhea, confusion, visual disturbances, and cardiac arrhythmias. Similar treatments for digoxin poisoning, such as the use of anti-digoxin antibody fragments, are also effective in digitoxin toxicity.

Medigoxin is a cardiac glycoside related to digoxin and digitoxin with the systematic name: 4-[(3S,5R,8R,9S,10S,12R,13S,14S)-12,14-Dihydroxy-3-[(2R,4S,5S,6R)-4-hydroxy-5-[(2S,4S,5S,6R)-4-hydroxy-5-[(2S,4S,5S,6R)-4-hydroxy-5-methoxy-6-methyl-oxan-2-yl]oxy-6-methyl-oxan-2-yl]oxy-6-methyl-oxan-2-yl]oxy-10,13-dimethyl-1,2,3,4,5,6,7,8,9,11,12,15,16,17 tetradecahydrocyclopenta[a]phenanthren-17-yl]-5H-furan-2-one.

Lanatoside C is a cardiac glycoside with the systematic name: [6-[6-[6-[[12,14-dihydroxy-10,13-dimethyl-17-(5-oxo-2H-furan-3-yl)-1,2,3,4,5,6,7,8,9,11,12,15,16,17-tetradecahydrocyclopenta[a]phenanthren-3-yl]oxy]-4-hydroxy-2-methyloxan-3-yl]oxy-4-hydroxy-2-methyloxan-3-yl]oxy-2-methyl-3-[3,4,5-trihydroxy-6-(hydroxymethyl)oxan-2-yl]oxyoxan-4-yl]acetate.

Proscillaridin is a bufanolide cardiac glycoside obtainable from plants of the genus Scilla. The systematic name of this compound is: 3β-Rhamnosido-14β-hydroxybufa 4, 20, 22 trienolide.

K-strophanthin refers to a cardiac glycoside or mixture of glycosides obtained from a tropical plant (Strophanthus kombé) of the dogbane family.

Peruvoside refers to a cardiac glycoside with the systematic name: 6-[6-[6-[[12,14-dihydroxy-10,13-dimethyl-[7-(5-oxo-2H-furan-3-yl)-1,2,3,4,5,6,7,8,9,11,12,15,16,17-tetradecahydrocyclopenta[a]phenanthren-3-yl]oxy]-4-hydroxy-2-methyloxan-3-yl]oxy-4-hydroxy-2-methyloxan-3-yl]oxy-2-methyl-3-[3,4,5-trihydroxy-6-(hydroxymethyl)oxan-2-yl]oxyoxan-4-yl]acetate

Ouabain, also known as g-strophanthin, is a cardiac glycoside found in the ripe seeds of the African plants Strophanthus gratus and Acokanthera ouabaio. The systematic name of this compound is: 1β,3β,5β,11a,14,19-Hexahydroxycard-20(22)-enolide 3-(6-deoxy-a-L-mannopyranoside).

Other embodiments of cardiac glycosides include oleander and extracts and isolates thereof.

B. Calciotropic Agents

In some embodiments of the present invention, one or more calciotropic agents are used in combination with a cardiac glycoside. Examples of calciotropic agents known in the art include: Vitamin D3 (cholecalciferol), other Vitamin D3 analogues, PTH, lipid phosphatidylinositol, PTHrP, magnesium, thiazide diuretic, and lithium.

Vitamin D3 or cholecalciferol is a hormone which is essential for promoting calcium absorption in the gut and maintaining adequate serum calcium and phosphate concentrations to enable normal mineralization of bone and to prevent hypocalcemic tetany. Vitamin D is normally synthesized from 7-dehydrocholesterol in the skin. It is also naturally present in some foods (such as fish and fish liver oils, beef liver, cheese, and egg yolks), added as a supplement to foods such as milk or yogurt, or available as a dietary supplement, such as vitamin D pills.

Examples of vitamin D analogues include calcipotriene, a synthetic form of vitamin D3 approved by the FDA for treating psoriasis. Calcipotriene is commercially available under the brand name Dovonex, which is sold as a cream, ointment, and scalp solution. All Dovonex products come in 0.005% concentration. Examples of other vitamin D analogues are known in the art, such as those described in Steddon et al., Nephrol Dial Transplant 16: 1965-1967 (2001)

Parathyroid hormone (PTH), or parathormone, is a hormone secreted by the parathyroid glands as a polypeptide containing 84 amino acids. PTH acts to increase the concentration of calcium (Ca⁺²) in the blood. Examples of PTH preparations that may be used in the practice of the invention include those found in U.S. Pat. Nos. 5,496,801 and 5,407,911, herein incorporated by reference in their entireties.

Thiazide diuretics are a class of compounds derived from benzothiadiazine. They function as diuretics by inhibiting Na⁺/Cl− reabsorption from the distal convoluted tubules in the kidneys by blocking the thiazide-sensitive Na⁺-Cl⁻ symporter. Thiazides also lower urinary calcium excretion and thus promote a positive calcium balance. Examples of members of this class of compounds include: chlorothiazide sodium (Diuril) (systematic name: 6-chloro-2H-1,2,4-benzothiadiazine-7-sulfonamide 1,1-dioxide; cydrochlorothiazide (systematic name: 6-chloro-3,4-dihydro-2H-1,2,4-benzothiadiazine-7-sulfonamide 1,1-dioxide, available under a variety of brand names such as Apo-Hydro, Aquazide H, Dichlotride, Hydrodiuril, HydroSaluric, Microzide, Oretic; and bendroflumethiazide (3-benzyl-5,5-dioxo-9-(trifluoromethyl)-5λ⁶-thia-2,4-diazabicyclo[4.4.0]deca-7,9,11-triene-8-sulfonamide).

C. Vasoconstrictors

Any of a number of agents which cause vasoconstriction are known in the art and may be used in the practice of the present invention. Such agents may include: muscarinic agonists, e.g. acetylcholine; Neuropeptide Y (NPY), a 36 amino acid peptide neurotransmitter; adrenergic agonists, e.g. norepinephrine; thromboxane; endothelin; and angiotensin II. See, e.g., Goodman and Gilman's The Pharmacological Basis of Therapeutics, 11th Edition, 2006 for further examples.

D. Collagen Barriers

Collagen barriers to minimize the distribution of the cardiac glycosides and calciotropic agents of the invention may be formed from either type I or II collagen from bovine or porcine sources. They are often cross-linked and resorbed between 4-38 weeks depending on the type. Brands of collagen barriers include Biomend, Biomend Extend, OSSIX, Neomem, and Hypro-Sorb. Alternatively, barriers made of other resorbable materials may be used. Such synthetic membranes may be polymers of lactic acid or glycolic acid, containing ester bonds which are degraded over 30-60 days. Among the commercially available synthetic barriers include: Vicryl, Atrisorb, Atrisorb-FreeFlow, Arisorb-D, Resolut XT, Epi-Gide and Gore Resolut Adapt, each made predominantly of acid polymers. In addition, Capset is a calcium sulphate derivative synthetic membrane. In some instances, non-absorbable ePTFE membranes may be used to form barriers, although in general, resorbable barriers are preferred.

E. Agents for Topical Applications

Local (such as local infiltration) or topical application of the cardiac glycoside alone or in combination is envisaged. Specific techniques include topical application, local infiltration and iontophoresis which utilizes a DC current to “drive” molecules across the skin, The advantage of local or topical application is that potentially higher concentrations (supratherapeutic doses) of the cardiac glycoside can be used with less risk of systemic effects. Delivery using phospholipid—based vesicular (liposomal) systems has also been envisaged. Liposomal systems favor the retention of the drug at the affected site for a prolonged period of time (i.e., depot or reservoir effect).

Prodrug formulations of cardiac glycosides can be used to deliver higher local levels for a longer duration while avoiding systemic toxicity. Cardiac glycosides linked to an oligopeptide that can be cleaved by an enzyme that is present in the extracellular environment of target cells, a stabilizing or masking group and, optionally, a linker group is one category of a prodrug formulation. Another example involves protein-bound, for example, albumin-bound, cardiac glycosides for local injectable suspension.

A prodrug can be used for administration to a patient, wherein the prodrug is formed by linking an oligopeptide at a first attachment site of the oligopeptide to a stabilizing group, and directly or indirectly linking the oligopeptide to a cardiac glycoside at a second attachment site of the oligopeptide.

F. Other Agents

The compositions provided herein may include a cardiac glycoside and/or a calciotropic agent. The compositions also may include a thiol depleting agent.

G. U.V. Light

In certain embodiments, the calciotropic agent forms upon the administration of U.V. light. For example, a precursor of vitamin D can be administered, or provided in the composition, that will be converted to vitamin D by U.V. light.

Vitamin D 3 is a derivative of provitamin D 3 (7-dehydrocholesterol), the immediate biological precursor of cholesterol. When skin is exposed to sunlight or artificial sources of ultraviolet (UV) radiation, the UV radiation penetrates the epidermis and causes a variety of biochemical reactions. Included in these reactions is the transformation of provitamin D 3 to previtamin D 3. The solar electromagnetic energy having wavelengths between 290 and 315 nm is absorbed by provitamin D 3 resulting in its fragmentation to previtamin D 3. Although previtamin D 3 is biologically inert, it is thermally labile and spontaneously undergoes a temperature-dependent rearrangement to form the thermally stable vitamin D 3. After biosynthesis, vitamin D 3 is translocated from the epidermis into the circulation via a vitamin-D binding protein. Holick et al., Science 211:590-593 (1981); Holick et al. in Braunwald et al., Harrison's Principles of Internal Medicine, 11th ed., McGraw-Hill (1987), pp. 1857-69.

It has been disclosed (Holick, M., Transactions of the Association of American Physicians, 42:54-63 (1979); Molecular Endocrinology; Maclntyre and Szelke, eds.; Elsevier/North Holland Biomedical Press (1979), pp. 301-308) that the topical application of hydroxylated metabolites of provitamin D compounds to the skin combined with U.V. phototherapy is a method for the sustained administration of vitamin D metabolites to patients who suffer vitamin D metabolic disorders. When the hydroxylated provitamins are applied and irradiated with ultraviolet radiation, they convert to hydroxylated previtamins which then thermally isomerize to the hydroxylated vitamin D. This work is also disclosed in Holick et al., New England Journal of Medicine 301:349-354 (1980) and U.S. Pat. No. 4,310,511 (Jan. 12, 1982).

This in certain embodiments the compositions disclosed herein contain vitamin D precursors (such as ergosterol) which, when irradiated with ultraviolet rays, are transformed into vitamin D or an active analogue thereof. The wavelength for the production of previtamin D 3 can be, e.g., between 295 nm and 300 nm.

Compositions, including topical compositions, comprising lumisterol and tachysterol and derivatives thereof, can be used to allow low energy UV photoconversion of lumisterol and tachysterol and derivatives thereof to previtamin D and derivatives thereof as a method of producing vitamin D as described in U.S. Pat. No. 5,422,099, the disclosure of which is incorporated herein.

In certain embodiments, the U.V. light source is sunlight. In other embodiments, a U.V. lamp can be used.

H. Delivery

Optionally, the cardiac glycoside is delivered using a liposomal system. In another embodiment, the cardiac glycoside is delivered using bubble liposomes and ultrasound. In another embodiment, the cardiac glycoside is protein-bound and delivered via injectable suspension.

The active agents also can be delivered locally using mechanical barriers to limit diffusion of said compounds such as a balloon catheter to dam said drug within a blood vessel.

3. Administration

A. Dosing

Cardiac glycosides and calciotropic agents are administered in accordance with dosages and scheduling regimens as determined and practiced by those of skill in the art. General guidance for appropriate dosages of all pharmacological agents used in the present methods is provided in Goodman and Gilman's The Pharmacological Basis of Therapeutics, 11^{th} Edition, 2006, supra, in a Physicians' Desk Reference (PDR), for example, in the 62^{nd} (2008) Ed., Thomson PDR, and in the FDA Orange Book, which are hereby entirely incorporated by reference herein. In the compositions and methods of the present invention, in some embodiments, efficacious dosages of cardiac glycosides and calciotropic agents for practicing the present invention can be equal to or less than (e.g., about 25, 50, 75, or 100%) the dosages published for other indications. Combining a cardiac glycoside with a calciotropic agent can allow for both pharmacological agents to be administered at subtherapeutic doses and elicit an efficacious effect in treating, ameliorating, or preventing a hyperproliferative disease in a subject.

The appropriate dosage of cardiac glycosides and calciotropic agents will vary according to several factors, including the chosen route of administration, the formulation of the composition, patient response, the severity of the condition, the subject's weight, and the judgment of the prescribing physician. The dosage can be increased or decreased over time, as required by an individual patient. For example, an individual patient's dosages of a cardiac glycoside and calciotropic agents can be adjusted to achieve an optimal or anti-psoriasis effect while avoiding side effects such as heart irregularities.

The use of an agent that minimizes diffusion of cardiac glycosides and calciotropic agents from a region of the body where a therapeutic effect is desired allows higher dosages of these agents to be used. Thus, for example, even supra therapeutic doses of cardiac glycosides, with or without a calcitoropic agent, may be used if the cardiac glycoside is co-administered with an additional agent when restricts the distribution of the cardiac glycoside from a therapeutically relevant site (e.g., a psoriatic skin lesion) to an organ such as the heart where life threatening side effects could occur. Examples of agents which can serve as diffusion-limiting components include vasoconstrictors and collagen barriers as described herein. Similarly, where treatment is localized in any manner, then potentially higher doses can be used.

Determination of an effective amount is well within the capability of those skilled in the art, especially in light of the detailed disclosure provided herein. Applicable methods for determining an appropriate dose and dosing schedule for administration of the combinations of the present invention are described, for example, in Goodman and Gilman's The Pharmacological Basis of Therapeutics, 11^{th} Edition, 2006, supra, and in a Physicians' Desk Reference (PDR), supra, in Remington: The Science and Practice of Pharmacy, 21^{st} Ed., 2005 and in Martindale: The Complete Drug Reference, Sweetman, 2005, London: Pharmaceutical Press, and in Martindale, Martindale: The Extra Pharmacopoeia, 31st Ed., 1996, Amer. Pharma. Assn., each of which are hereby incorporated by reference herein.

As an example, for the treatment of heart conditions, digoxin is typically administered initially as a loading dose (“digitalization”), followed by the administration of maintenance doses. Because skeletal muscle serves as a reservoir of digoxin, dosing is based on estimated lean body mass, where lean body weight is defined as total body mass minus fat mass, which is generally determined with the use of skin calipers. Monitoring may be required during administration so that the target serum concentration is, e.g., about 1.0 ng/ml.

For the treatment of heart conditions, digoxin is usually given orally, and more rarely, intravenously. A loading dose of 15 mcg/kg of lean body weight can be administered in three divided doses at 6 hour intervals. Thus, for example, in an individual with a lean body weight of 50 kg, this would result in a total dose of 15×50=750 mcg. The total dose is divided by three to give an individual dose of 250 mcg. If a desired effect is not observed (e.g., a reduction of the ventricular rate to a desired target), an additional dose of 5 mcg/kg can be given, providing there are no symptoms or signs of toxicity.

For heart treatments, the maintenance dose is calculated as a fraction of the effective loading dose, adjusted for renal function as shown in the example below.

TABLE 1
Digoxin maintenance doses

| | Daily maintenance dose as |
|---|---|
| | a fraction of the |
| Creatinine clearance (ml/min) | effective loading dose |
| 100 | ⅓ |
| 50 | ¼ |
| 25 | ⅕ |
| 10 | ⅙ |
| 0 | 1/7 |

In some embodiments, the cardiac glycoside is administered at a dose that is supra therapeutic. It is generally accepted that the toxic effects of cardiac glycosides occur in a range exceeding the therapeutic dose for human therapy of heart conditions by a factor of 1.5 to 3. Accordingly, in one embodiment of this invention, cardiac glycosides are administered to subjects having a hyperproliferative disorder at a dose which exceeds the therapeutic dose by a factor equal or greater than 1.5. In another embodiment, cardiac glycosides are administered to subjects having a hyperproliferative disorder at a dose which exceeds the therapeutic dose by a factor equal or greater than 3. The cardiac glycoside may even be locally administered in an amount that would be potentially lethal if systemically administered.

In other embodiments, a cardiac glycoside is administered at a dose which is sub-therapeutic as compared to when the cardiac glycoside is administered alone. As an example, standard maintenance doses for digoxin and digitoxin are 2.5 ng/ml for digoxin and 9-35 ng/ml for digitoxin. Thus, the doses should be adjusted to maintain a plasma concentration of less than 2.5 ng/ml for digoxin or less than 9-35 ng/ml for digitoxin to achieve subtherapeutic doses. In other embodiments (e.g., for the treatment of psoriasis), subtherapeutic doses are achieved, at least in part, due to the absence of the administration of a loading dose as described above.

In yet other embodiments, the cardiac glycoside is administered at standard therapeutic doses, and/or via established routes of administration and scheduling.

The cardiac glycoside can in some embodiments is provided

B. Scheduling

Generally, in practicing the present methods, effective amounts of a cardiac glycoside are administered alone or in combination with at least one or more calciotropic agents and/or one or diffusion-limiting components, such a vasoconstrictor or collagen barrier. Co-administered pharmacological agents can be administered together or separately, simultaneously or at different times. When administered, the cardiac glycoside alone or in combination with at least one or more calciotropic agents and/or diffusion-limiting components, can be administered once, twice, three, four times daily or more or less often, as needed. Preferably, the administered pharmacological agents are administered once daily. Preferably, the administered active agents are administered at the same time or times, for instance as an admixture. One or more of the pharmacological agents can be administered in a sustained-release formulation.

C. Routes of Administration

Administration of a cardiac glycoside alone or in combination with at least one or more calciotropic agents and/or a diffusion-limiting component, such a vasoconstrictor or collagen barrier, can be achieved in various ways, including oral, buccal, parenteral, intravenous, intradermal, subcutaneous, intramuscular, transdermal, transmucosal, intranasal, etc., administration. A pharmacological agent can be administered by the same or by a different route of administration when co-administered with another of the pharmacological agents of the invention.

In various embodiments, one or more of the pharmacological agents of the invention can be administered in a local rather than systemic manner, for example, transdermally or via another route in a depot or sustained release formulation. In other embodiments, one or more of the pharmacological agents of the invention can be administered orally. In yet other embodiments, one or more of the pharmacological agents of the invention can be administered sublingually.

D. Compositions

The present invention further provides pharmaceutical compositions comprising a mixture of an effective amount of a cardiac glycoside alone or in combination with at least one or more calciotropic agents and/or a diffusion-limiting component. In the compositions of the invention, the cardiac glycoside alone or in combination with at least one or more calciotropic agents and/or a diffusion-limiting component can be included in therapeutic, subtherapeutic, or supra therapeutic doses. In some embodiments, the compositions comprise one or both pharmacological agents in subtherapeutic doses.

An effective amount of a cardiac glycoside alone or in combination with at least one or more calciotropic agents and/or a diffusion-limiting component of this invention can be incorporated into a variety of formulations for therapeutic administration. More particularly, the pharmacological agents of the present invention can be formulated into pharmaceutical compositions, together or separately, by formulation with appropriate pharmaceutically acceptable carriers or diluents, and can be formulated into preparations in solid, semi-solid, liquid or gaseous forms such as tablets, capsules, pills, powders, granules, dragees, gels, slurries, ointments, solutions, suppositories, injections, inhalants, and aerosols.

Suitable formulations for use in the present invention are found in, for example, in Remington: The Science and Practice of Pharmacy, 21^{st} Ed., 2005; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated by reference herein. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

The pharmaceutical preparations of the present invention can be prepared for delivery in a sustained-release, controlled release, extended-release, timed-release or delayed-release formulation, for example, in semi-permeable matrices of solid hydrophobic polymers containing the effective agent(s). Various types of sustained-release materials have been established and are well known by those of skill in the art. Current extended-release formulations include film-coated tablets, multiparticulate or pellet systems, matrix technologies using hydrophilic or lipophilic materials and wax-based tablets with pore-forming excipients (see, for example, Huang, et. al., Drug Dev. Ind. Pharm. 29:79 (2003); Pearnchob, et. al., Drug Dev. Ind. Pharm. 29:925 (2003); Maggi, et. al., Eur. J. Pharm. Biopharm. 55:99 (2003); Khanvilker, et. al., Drug Dev. Ind. Pharm. 228:601 (2002); and Schmidt, et. al., Int. J. Pharm. 216:9 (2001). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidine (PVP); carboxyvinyl hydrophilic polymers; hydrophobic and/or hydrophilic hydrocolloids, such as methylcellose, ethylcellulaose, hydroxypropylcellulose, and hydroxypropylmethylcellulose; and carboxypolmethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering an effective amount of a cardiac glycoside alone or in combination with at least one or more calciotropic agents and/or a diffusion-limiting component of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, a cardiac glycoside alone or in combination with at least one or more calciotropic agents and/or a diffusion-limiting component can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, a cardiac glycoside alone or in combination with at least one or more calciotropic agents and/or a diffusion-limiting component can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Systemic or local administration can also be by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include creams, lotions, gels, ointments or transdermal delivery systems, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in a conventional manner.

In addition to the formulations described previously, a cardiac glycoside alone or in combination with at least one or more calciotropic agents and/or a diffusion-limiting component of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The cardiac glycoside and/or calciotropic agent can be localized, e.g., by application via a patch or by cleavable bond to a localizing polymer, and can optionally be associated with a cosmetic pigment such as a skin colored, green or yellow pigment. The patch can be, for example, formed for release of active agents when bent or distorted, and/or suitable for controlled release or differential release and absorption in different sections of the patch. The cardiac glycoside and/or calciotropic agent can be covalently attached to a suitable polymeric material, such as a lipid, cellulose derivative, gelatin, or polyethylene glycol. The covalent bond can be enzymatically or hydrophobically cleavable such as an amide, ester carbonate, or carbamate.

A cardiac glycoside alone or in combination with at least one calciotropic agent and/or diffusion limiting component can also be formulated as a topical cream or formulated into a patch. The compounds can be attached by covalent bonding to hydropohobic materials such as a fatty acid, triglyceride or derivatives, emulsions or acceptable oils. The hydrophobic materials are attached to the cardiac glycoside by covalent bonds that are not readily cleavable enzymatically or hydrolytically. Covalent bonds are for example, ethers, thioether, carbon-carbon (single, double, triple), and heterocycle. This allows actives to be present locally and to reduce or avoid systemic release.

A cardiac glycoside alone or in combination with at least one calciotropic agent and/or diffusion limiting component can also be formulated as a derivative that is active when applied, e.g., as a topical cream or transdermal patch. After absorption of the derivative, the derivative exerts a local effect but is rapidly deactivated by cleavage of the derivative group on systemic exposure leading to rapid excretion and cleavage. This is known as the “soft drug” approach. See e.g. Yang et al 1995, Pharm. Research 12:329-36. An example is Esmolol, a short acting compound cleaved by esterases.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The pharmaceutical compositions can have a suitable dose amount of cardioglycoside and optionally the calciotropic agent. For example, a solid or liquid composition may include a dose amount of about 0.001-500 mg cardioglycoside, such as digoxin, or, e.g., about 0.01-10 mg; 0.01-1 mg; about 0.01 to 0.5 mg, or e.g. about 0.1-1.0 mg. The calciotropic agent may be provided in the same or different composition in a dose amount, e.g., of about 1 mcg to 1 g calciotropic agent such as vitamin D, or e.g., about 1 to 1000 mcg, or about 1 to 100 mcg, or about 1-50 mcg.

The pharmaceutical compositions can have a suitable dose amount of cardioglycoside and optionally the calciotropic agent per gram composition. For example, a solid or liquid composition may include an amount of the cardiac glycoside of about 1 μg/g to 1 g/g; or about 1 μg/g-500 mg/g; or about 1 μg/g to 100 mg/g; or about 1 μg/g to 1 mg/g; or about 1 μg/g to 500 μg/g or about 1 μg/g to 200 μg/g. The calciotropic agent, such as vitamin D, may be provided in the same or different composition in an amount, e.g., of about 1 mcg/g to 1 g/g, or e.g., about 1 to 1000 mcg/g, or about 1 to 100 mcg/g, or about 1-50 mcg/g.

E. Kits

The pharmaceutical compositions of the present invention can be provided in a kit.

In some embodiments, a kit of the present invention comprises a cardiac glycoside alone or in combination with at least one or more calciotropic agents and/or a diffusion-limiting component in separate formulations. In other embodiments, the kits comprise a cardiac glycoside alone or in combination with at least one or more calciotropic agents and/or a diffusion-limiting component within the same formulation.

In some embodiments, the kits provide the cardiac glycoside alone or in combination with at least one or more calciotropic agents and/or a diffusion-limiting component independently in uniform dosage formulations throughout the course of treatment. In some embodiments, the kits provide a cardiac glycoside alone or in combination with at least one or more calciotropic agents and/or a diffusion-limiting component independently in graduated dosages over the course of treatment, either increasing or decreasing, to an efficacious dosage level, according to the requirements of an individual. The kits also may include instructions, e.g., on dosage or administration.

The following examples are illustrative but not limiting.

### EXAMPLES

### Example 1

A patient was diagnosed with chronic plaque psoriasis and psoriatic arthritis. She had been treated with multiple different regimens (topicals, phototherapy, antinflammatories, and biologics) without significant improvement. All other treatments were stopped and digoxin 0.25 mg with 800 IU of vitamin D was started. At her next monthly visit the psoriatic plaques had significantly regressed and the psoriatic arthritis had improved as well. The patient noted increased fatigue from the digoxin but otherwise no other side effects.

### Example 2

Two siblings with severe rheumatoid arthritis characterized by progressive pain, stiffness and swelling of joints in spite of treatment with NSAIDs, narcotics, and immunosuppressants were given 0.125 mg of digoxin daily. No response was noted until the 5^{th} day of treatment at which time both patients reported about a 10% improvement in pain levels and functionality. 2 weeks later the dose of digoxin was doubled to 0.25 mg and the patients both noted a proportional improvement in pain levels and functionality to 20%. No side effects from the digoxin were reported.

### Example 3

Digoxin and vitamin D for wound healing. A patient developed a 5×4 cm painful ulcer with elevated borders around an erythematous base, overlaid with a thick eschar with purulent exudate on her right elbow. Culture of purulent material however was not definitive. The patient was tried on 1 week of a cephalosporin and 1 week of an azole (fluconazole) with no change in the affected area. All treatment was stopped and the patient was placed on 0.125 mg of digoxin. Within 3 days the area had regressed by about 20% with healing that started in the center of the lesion. At this point vitamin D 1800 mg was started and healing of the area seemed to accelerate to the point that the wound seemed to have regressed about 25%. However, the wound healing seemed to plateau at this point in spite of increasing the dose of digoxin to 0.25 mg and the dose of vitamin D to 3600 IU. Consequently, the patient was scheduled for excision and biopsy of the wound.

### Example 4

Digoxin and vitamin D for hyperproliferative disorders: A patient presented with severe psoriasis characterized by plaques with a thin, silvery-white scale symmetrically distributed over the body and tender, inflamed ankle joints from psoriatic arthritis. His therapeutic regimen for the prior 4 months was a combination of topical agents (calcipotriene), phototherapy, and Methotrexate 5-10 mg q week along with NSAIDs for the joint pain. This combined approach reduced the severity and number of his psoriatic plaques and improved his joint pain but did not completely eliminate them. Methotrexate seemed to be moderately effective but made the patient quite nauseated and necessitated frequent dose adjustments. The patient was told to stop all other therapeutic modalities and he was started on 0.125 IU of digoxin for one month. The patient reported no side effects from the digoxin. At his next monthly visit the skin lesions had all disappeared and his joint pain had abated. As follow up, this patient remained on 0.125 mg of digoxin and although the psoriasis had still largely regressed the patient began to experience a mild recrudescence on his elbows and ankles 2 months later with return of joint pain in these areas. At this point the patient was started on 2000 IU of vitamin D per day and within 1 week the plaques on his elbows and ankles had disappeared along with the joint pain.

### Example 5

The patient presented with hereditary ichthyosis vulgaris characterized by symmetrical scaling of the skin and hyperkeratosis of the palms and soles resulting in fissuring. The patient was applying topical retinoids and lactic acid lotion to his skin for over 6 months with a mild but stable improvement. Digoxin 0.125 mg+vitamin D 1800 IU was added to his treatment regimen and the patient noted that the ichthyosis and scaling seemed to improve about 10%. On physical exam, scaling on the patient's upper extremities was noticeably improved while on the back there was very little improvement

### Example 6

The patient presented with perianal cellulitis characterized by carbuncles with warmth, erythema, edema, and tenderness of the affected area. The night before the patient's wife had sterilized a needle and expressed pockets of pus from the carbuncles so that the next day, on physical exam, the areas, while red and tender, were not abscessed. The patient was started on 0.125 mg digoxin and 1800 IU of vitamin D. Within 3 days after beginning treatment with digoxin and vitamin D the areas were no longer red and tender.

### Example 7

A patient presented with large, painful nodules in the axilla which he had drained with an unsterilized needle the night before. On exam the nodules were hard, red and painful but no longer raised or fluctuant. The patient was started on 0.125 mg digoxin and 1800 IU of vitamin D and within 2 days the lesions had resolved.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a hyperproliferative disorder in a subject, the method comprising administering to the subject an anti-proliferation effective amount of a cardiac glycoside and at least one calciotropic agent

2. The method of claim 1, wherein the hyperproliferative disorder is psoriasis or cancer.

3. The method of claim 1, wherein the hyperproliferative disorder is selected from the group consisting of disorders of keratinization and keratosis, diabetic retinopathy, endometriosis, macular degenerative disorders, keloids, warts, cirrhosis, chronic inflammatory-related disorders, proliferative vitreoretinopathy, retinopathy of prematurity, granulomatosis, immune hyperproliferation associated with organ or tissue transplantation, benign prostatic hypertrophy and an immunoproliferative disease or disorder.

4. The method of claim 1, wherein the administration of the at least one calciotropic agent results in a transient or sustained rise in calcium levels.

5. The method of claim 1, wherein the calciotropic agent is selected from the group consisting of: Vitamin D3 (cholecalciferol), a Vitamin D3 analogue, PTH, lipid phosphatidylinositol, PTHrP, magnesium, thiazide diuretic, and lithium.

6. The method of claim 1, wherein the Vitamin D3 analogue is calcipotriene (Dovonex).

7. The method of claim 1, wherein the cardiac glycoside is selected from the group consisting of: digoxin, digitoxin, medigoxin, lanatoside C, proscillaridin, K strophanthin, peruvoside, and ouabain.

8. The method of claim 1, wherein the cardiac glycoside is covalently or nocovalently attached to a targeting or stabilizing group that is optionally cleavable and is optionally PEG or albumin; a lipophilic moiety that is optionally a fatty acid amide or triglyceride that slows systemic exposure after topical application; or a group that is cleaved on system exposure leading to deactivation of the drug and rapid clearance from systemic circulation.

9. The method of claim 1, wherein the cardiac glycoside is administered at a dose of about 0.1 to 1000 mg/kg of the patient's weight.

10. The method of claim 1, further comprising the step of administering capsaicin or capsaicin congeners and derivatives optionally selected from resiniferatoxin, Capsinolol, and N-arachidonoyldopamine (NADA).

11. The method of claim 1, further comprising the step of administering at least one agent selected from the group consisting of a vasoconstrictor that is optionally epinephrine, a cell depolarizing agent, a pain-reducing agent, a chemotherapeutic agent, an anti-angiogenic agent, a radiosensitizer, a pain-reducing agent, a diffusion-limiting component, and calcium.

12. The method of claim 1, comprising local or topical administration optionally utilizing mechanical barriers to limit diffusion of said compounds.

13. The method of claim 1, further comprising the step of administering a diffusion-limiting component that optionally comprises collagen, and optionally further comprising administering calcium.

14. The method of claim 1, wherein said cardiac glycoside is administered at a sub-therapeutic dose that optionally results in a plasma concentration of less than 2.5 ng/ml for digoxin or less than 9-35 ng/ml for digitoxin.

15. The method of claim 1, wherein said cardiac glycoside is administered at a supra therapeutic dose optionally at least 1.5 times greater or at least 3 times greater than a therapeutic dose for treatment of heart conditions.

16. (canceled)

17. A method of treating pain resulting from a hyperproliferative disorder in a subject, the method comprising administering to the subject an anti-proliferation effective amount of a cardiac glycoside and at least one calciotropic agent.

18. The method of claim 17, wherein said hyperproliferative disorder is selected from the group consisting of: psoriatic arthritis, rheumatoid arthritis, lupus, reactive arthritis, Sjogren's disease, inflammatory bowel disorder, dermatomyositis, ankylosing spondylitis, juvenile rheumatoid arthritis, gout, inflammatory osteoarthritis, pseudogout, and amyloidosis.

19-31. (canceled)

32. A pharmaceutical composition for the treatment of a hyperproliferative disorder or pain associated therewith in a subject comprising an anti-proliferation effective amount of a cardiac glycoside and at least one calciotropic agent.

33. (canceled)

34. The pharmaceutical composition of claim 32, wherein said calciotropic agent is selected from the group consisting of: Vitamin D3 (cholecalciferol), a Vitamin D3 analogue, PTH, lipid phosphatidylinositol, PTHrP, magnesium, thiazide diuretic, and lithium.

35-39. (canceled)

40. A kit for the treatment of a hyperproliferative disorder, said kit comprising a cardiac glycoside, a calciotropic agent, and a diffusion limiting component.

41-42. (canceled)
